(19) United States
(12) Patent Application Publication
(10) Pub. No.: US 2011/0244482 A1
(43) Pub. Date: Oct. 6, 2011

(54) ASSESSMENT OF SUBCHONDRAL BONE REMODELLING BY MEASURING CATHEPSIN K FRAGMENTS OF COLLAGEN TYPE II

(75) Inventors: Diana J. Leeming, Copenhagen (DK); Inger Byrjalsen, Hoersholm (DK); Per Qvist, Klampenborg (DK); Morten A. Karsdal, Copenhagen (DK)

(73) Assignee: NORDIC BIOSCIENCE A/S

(21) Appl. No.: 13/128,929

(22) PCT Filed: Nov. 11, 2009
(86) PCT No.: PCT/EP09/64991

§ 371 Date: Jun. 23, 2011

(30) Foreign Application Priority Data
Nov. 13, 2008 (GB) 0820785.4

(51) Int. Cl. G01N 33/566; C07K 16/18

(52) U.S. Cl. 435/7.9; 436/501; 530/389.1; 530/387.9

(57) ABSTRACT

A method of assay to determine the extent of collagen type II resorption activity comprising measuring the level of fragments of collagen type II that contain a cathepsin K generated neo-epitope not shared by collagen type I by binding the neo-epitope with an antibody specific for the neo-epitope and detecting the level of binding of said binding partner.

The present invention relates to methods of immunoassay of peptide fragments generated by proteolytic cleavage of type II collagen by Cathepsin K which are excellent biomarkers of subchondral bone remodelling.

Osteoarthritis is one of the leading causes of disability in the world, with more than 10% of the elderly population having symptomatic disease (Woolf & Pfleger, 2003). The incidence increases with age, and by age 65, 80% has radiographic evidence of OA (Lawrence et al., 1998). Therefore, osteoarthritis is both prevalent and a serious burden to the patient and the society. However, at present there is little to offer the affected individuals for prevention of the disease or treatment in the early stages. For many patients, hip or knee replacement is eventually the only treatment option.

Although the pathogenicity of osteoarthritis is not fully understood at present, it is evident that a central hallmark in this slow, chronic disease is progressive destruction of the articular joints, which consists of bone, cartilage and the synovium. In particular the cartilage has attracted much attention, and the different grades and stages of OA cartilage histopathology have recently been detailed described by a working group under OARSI (Pritzker et al., 2006). This system encompasses 7 grades (or severity levels) with involvement of the deeper cartilage layers in more advanced OA disease, and when combined with the extent of cartilage involvement expressed in 5 stages this leads to a semi-quantitative scoring system of 0 to 24.

However, the lack of sensitive, specific and fast analytical techniques to assess important metabolic processes in articular cartilage and their effects on the structure of the tissue is a major barrier to effective drug development in OA.

In particular, major efforts have been allocated to the development of new and better biochemical markers of cartilage turnover.

Cartilage, including articular cartilage, is for the most part composed of collagen type II (60%-70% of dry weight) and proteoglycans (10% of dry weight). Cartilage degradation is mainly mediated by the MMPs and the closely related ADAM-TS (a disintegrin and metalloproteinase with thrombospondin motifs), but collagen type II is most sensitive to MMP activity (Dean et al., 1989; Reboul et al., 1996; Hui et al., 2003). The action of these proteases results in the release of various extracellular fragments which could be candidates as biomarkers of cartilage degradation.

Since type II collagen is the most abundant protein in cartilage, several different degradation fragments of collagen type II have been indicated as useful for monitoring degenerative diseases of the cartilage (Schaller et al., 2005; Sumer et al., 2006; Birmingham et al., 2006). A fragment of the C-telopeptide of type II collagen, i.e. CTX-II, is generated by MMP-activity (Christgau et al., 2001; Mouritzen et al., 2003) and measurement of CTX-II has been reported for monitoring degradation of type II collagen in experimental setups assessing cartilage degradation (Schaller et al., 2005) as well as in humans (Reijman et al., 2004).

However, the first report of using antibodies for detection of collagen type II fragments came from Billinghurst and co-workers (Billinghurst et al., 1997), who described detection of an amino-terminal neoepitope on the shorter fragment of type II collagen after cleavage by collagenase.

More well-described in the literature is the C2C neoepitope at the C-terminus of the ¾ length fragment (Fraser et al., 2003; Poole et al., 2004). This test is dependant of the binding of a monoclonal antibody to the amino acid sequence EGPP(OH)GPQG SEQ ID NO:1 (Poole et al., 2004).

Other tests for collagen type II fragments include the C1,2C, which, however, is based on the amino acid sequence GPP(OH)GPQG SEQ ID NO:2 found in both type I and type II (Billinghurst et al., 1997). Also, another fragment generated by the action of collagenase is the TIINE fragment (Otterness et al., 1997) and can be detected using monoclonal antibody 9A4 recognising the neoepitope Gly-Pro-Pro-Gly-Pro-Gln-Gly-COOH SEQ ID NO:3. Combined with monoclonal antibody 5109 (Downs et al., 2001) as a capture antibody, the sandwich test is claimed to be specific for type II collagen.

Apart from the involvement of articular cartilage in OA, an increasing body of evidence demonstrates that skeletal structural integrity is a prerequisite for preventing progression of joint disease.

Examinations of peri-articular bone in knees and hips of patients with OA have confirmed that the subchondral bone is abnormal in OA joints, with altered trabecular structure and sclerosis of the subchondral plate (Hunter et al., 2003A; Hunter et al., 2003B). Bone scintigraphy has revealed localization of the nuclide to sites of increased subchondral bone turnover (Dieppe et al., 1993). Cross-sectional studies have also established that women with advanced knee or hip OA have higher bone mineral densities (BMD) near, or at the site of joint OA (Arden et al., 2006). In fact, subchondral bone turnover has been shown to be as much as 20 fold increased compared to that of normal bone turnover (Bailey et al., 2004).

Subchondral bone is separated from the articular cartilage only by a layer of calcified cartilage (Burr et al., 2003). This allows for several possibilities of transmitting signals from one compartment to the other. Among these are increased vascularisation, and the development of microcracks occurring in the bone matrix, both phenomena which have been strongly implicated in initiation of bone remodelling, as well as increased degradation of the calcified cartilage (Burr et al., 2003; Lajeunesse et al., 2003).

Despite the fact that subchondral bone abnormality could be an important risk factor for the development of OA, the metabolic activity in this compartment cannot be studied with biochemical markers, as markers with sufficient specificity has not been identified.

However, we have discovered that the quantification of fragments of type II collagen carrying neo-epitopes generated by the proteolytic cleavage of the protein by cathepsin K are both sensitive and specific markers of subchondral bone turnover. Furthermore we have discovered that elevated levels of these fragments in body fluids is associated with a high risk for progression of OA and therefore could be incorporated into the overall risk assessment of this disease. It is conjectured that such fragments are produced when calcified cartilage is exposed to cathepsin K produced by osteoclasts.

Cathepsin K cleavage of type II collagen has been reported in a few studies. The first study to associate cathepsin K with cleavage of type II collagen was reported by Kafienah et al. (1998). Kafienah and coworkers described helical cleavage site(s) of collagen type II by Cathepsin K, and provided the amino acid sequence for one cleavage site, i.e.

| | |
|---|---|
| PGDDGEAGKPG [symbol] KSGERGPPG | SEQ ID NO: 4 |
| (bovine sequence) | |

Ten years later, Dejica et al. (2008) reported the development of an enzyme-linked immunosorbent assay based on polyclonal antibodies against the C-terminal neoepitope (C2K) of the cathepsin K cleavage site reported by Kefienah, i.e.

| | |
|---|---|
| PGDDGEAGKPG [symbol] KAGERGPPG | SEQ ID NO: 5 |

As the seven C-terminal amino acids of this epitope, i.e. GEAGKPG SEQ ID NO:6 can be found in type I collagen as well, this test will not distinguish type I and type II collagen fragments generated by cathepsin K activity. In contrast, according to the present invention such sequences are de-selected to increase specificity for type II collagen fragments. The measure in Dejica is not taught to provide information regarding subchondral bone resorption.

U.S. Pat. No. 6,642,007 (Saltarelli) disclose methods for monitoring urine for type II collagen fragment using a combination of a capture antibody and a detection antibody, such that type II collagen is distinguished from other collagen fragments.

U.S. Pat. No. 6,030,792 (Otterness) discloses antibodies for detecting collagen type II fragments resulting from collagenase cleavage. In particular, the following sequences are disclosed;

| | |
|---|---|
| GPPGPQG | SEQ ID NO: 7 |
| GEPGDDGPSG | SEQ ID NO: 8 |
| APGEDGRPGPPGP | SEQ ID NO: 9 |
| GKVGPSGAPGEDGRPG | SEQ ID NO: 10 |
| AEGPPGPQG | SEQ ID NO: 11 |
| GPPGPQGLAG | SEQ ID NO: 12 |
| GEPGDDGPS | SEQ ID NO: 13 |
| GEPGDDGPSGAEGPPG | SEQ ID NO: 14 |
| EKGEPGDDAPSGAEGPPGPQG | SEQ ID NO: 15 |
| GPPGPPGKPGDDGEAGKPGKA | SEQ ID NO: 16 |
| GPPGPRGRSGETGPAGPPGNP | SEQ ID NO: 17 |
| GAPGPQGFQGNPGEPGEPGVSY | SEQ ID NO: 18 |
| GEPGDDAGPSGAEGPPGPQG | SEQ ID NO: 19 |

A series of patents (U.S. Pat. Nos. 6,602,980; 6,566,492; 6,348,320; 6,255,056; 6,153,732; 6,143,511; 6,100,379; 5,919,634; 5,702,909; 5,688,652; 5,641,837; 5,641,687; 5,532,169; and 5,455,179) (Eyre) relates to peptides and methods for cartilage resorption assays employing antibodies binding to epitopes in the telopeptides of type II collagen.

U.S. Pat. No. 5,283,197 (Robins) describes methods of detecting collagen fragments cross-linked with lysyl pyridinoline or hydroxylysyl pyridinoline.

U.S. Pat. No. 7,410,770 (Reginster) disclosed methods for detection of collagenase-generated fragments of collagen type II using antibody binding to epitope in the amino acid sequence HRGYPGLDG SEQ ID NO:20 located in the helical region of collagen type II.

U.S. Pat. No. 6,132,976 (Poole) discloses methods for detecting cartilage degradation using antibodies which does not bind to unwound (native) type II collagen fragments but only to fragments being generated by collagenase cleavage. In particular the following amino acid sequences originating from type II collagen are included;

| | |
|---|---|
| CGKVGPSGAPGEDGRPGPPGPQY | SEQ ID NO: 21 |
| APGEDGRPGPPGP | SEQ ID NO: 22 |
| GQPG | SEQ ID NO: 23 |
| GPPGPQG | SEQ ID NO: 7 |
| CGGEGPPGPQG | SEQ ID NO: 24 |
| GAEGPPGPQGLAGQRGIVG | SEQ ID NO: 25 |
| GAPGTPGPQGIAGQRGVVG | SEQ ID NO: 26 |
| GPPGTPGPQGLLGAPGILG | SEQ ID NO: 27 |
| GPPGAPGPLGIAGITGARG | SEQ ID NO: 28 |
| CGGEGPPGPQGL | SEQ ID NO: 29 |
| CGGEGPPGPQGLA | SEQ ID NO: 30 |
| CGGEGPPGPQ | SEQ ID NO: 31 |
| CGGEGPPGP | SEQ ID NO: 32 |
| CGPPGPQG | SEQ ID NO: 33 |

U.S. Pat. No. 7,115,378 (Welsch) describes the use of mass spectrometry for identifying and quantifying peptides resulting from enzyme cleavage of collagen type II. The technique is used for identification and quantification of the peptides in a biological sample to assess activity of proteolytic enzymes in osteoarthritis and rheumatoid arthritis. In particular, the following sequences originating from humans are disclosed;

SEQ ID NO: 7
GPPGPQG
SEQ ID NO: 34
LQGPAGPPGEKGEPGDDGPSGAEGPPGPQG
PQG
SEQ ID NO: 35
PGPQG
SEQ ID NO: 36
PPGPQG
SEQ ID NO: 37
VLQGPAGPPGEKGEPGDDGPSGAEGPPGPQG
SEQ ID NO: 38
KGARGDSGPPGRAGEPGLQGPAGPPGEKGEPGDDGPSGAEGPPGPQG
SEQ ID NO: 39
ARGDSGPPGRAGEPGLQGPAGPPGEKGEPGDDGPSGAEGPPGPQG
SEQ ID NO: 40
GPAGPPGEKGEPGDDGPSGAEGPPGPQG
SEQ ID NO: 41
GPIGPPGERGAPGNRGFPGQDGLAGPKGAPGERGPSGLAGPKGANGD
PGRPGEPGLPGARGLTGRPGDAGPQGKVGPSGAPGEDGRPGPPGPQG
ARGQPGVMGFPGPKGANGEPGKAGEKGLPGAPGLGLPGKDGETGAEG
PPPA . . .

U.S. Pat. No. 6,706,490 (Cook) describes the detection of antibodies to collagen using CB peptides, in particular CB10, of mammalian type II collagen. Cyanogen bromide cleaves the carboxyl terminal of methionine residues thereby producing the CB peptides. The CB10 peptide has the sequence:

SEQ ID NO: 42
MPGERGAAGIAGPKGDRGDVGEKGPEGAPGKDGGRGLTGPIGPPGPAG
ANGEKGEVGPPGPAGSAGARGAPGERGETGPPGTSGIAGPPGADGQPG
AKGEQGEAGQKGDAGAPGPQGPSGAPGPQGPTGVTGPKGARGAQGPPG
ATGFPGAAGRVGPPGSNGNPGPPGPPGPSGKDGPKGARGDSGPPGRAG
EPGLQGPAGPPGEKGEPGDDGPSGAEGPPGPQGLAGQRGIVGLPGQRG
ERGFPGLPGPSGEPGQQGAPGASGDRGPPGPVGPPGLTGPAGEPGREG
SPGADGPPGRDGAAGVKGDRGETGAVGAPGAPGPPGSPGPAGPTGKQG
DRGEAGAQGPM

U.S. Pat. No. 7,195,883 (Rosenquist) disclose sandwich immunoassays in which a single antibody specific for the amino acid sequence EKGPDP SEQ ID NO:43 is used to detect telopeptide fragments fo type II collagen.

U.S. Pat. Nos. 6,420,125 and 6,107,047 (Fledelius) disclose methods of measuring the rate of degradation of collagen using antibodies binding to an amino acid sequence of type II collagen containing an isoaspartic acid residue. Also, the use of synthetic peptides having an amino acid sequence of type II collagen that contains an isoaspartic acid residue is described. In particular, the following sequences from type II collagen are described;

| | |
|---|---|
| GDIK*DIV | SEQ ID NO: 44 |
| EKGP*D, | SEQ ID NO: 45 |

where (*) denotes an isomerised peptide bond.

U.S. Pat. No. 6,300,083 (Fledelius) describes the determination of the amount of a D-amino acid containing fragment of the protein in a body fluid using an antibody capable of discriminating between the D-amino acid containing fragment and its L-amino acid containing analogue. In particular, the application includes the following peptide sequences from type II collagen;

| | |
|---|---|
| GDIKDIV | SEQ ID NO: 46 |
| EKGPD | SEQ ID NO: 43 |

U.S. Pat. Nos. 6,372,442 and 6,210,902 (Bonde) describes methods of characterizing the degradation of type II collagen. At least two distinct immunological assays should be used, each using a different immunological binding partner, and a numerical index is formed representing the difference in the results of the assays. In particular, the following type II collagen sequences are disclosed;

| | |
|---|---|
| EKGPDP | SEQ ID NO: 47 |
| EKGPD | SEQ ID NO: 43 |
| GVK | |
| PGVKG | SEQ ID NO: 48 |
| PGPKGE | SEQ ID NO: 49 |
| GQKGEP | SEQ ID NO: 50 |
| GDIKDIV | SEQ ID NO: 46 |

U.S. Pat. Nos. 6,355,442; 6,342,361; 6,323,314 and 6,110,689 (Qvist) describe the use of antibodies recognizing synthetic peptides for detection of collagen fragments. In particular, the following amino acid sequences are included;

| | |
|---|---|
| PGPKGE | SEQ ID NO: 49 |
| GQKGEP | SEQ ID NO: 50 |
| GDIKDIV | SEQ ID NO: 46 |
| EKGPD | SEQ ID NO: 43 |
| GVK | |
| PGVKG | SEQ ID NO: 48 |

U.S. Pat. No. 6,010,863 (Te Koppele) discloses the use of a sandwich immunoassay for the detection of collagen degradation using a first antibody directed at an epitope present on a collagen molecule at a distance of up to 165 amino acids from a collagen telopeptide crosslink site, and a second antibody directed at another epitope of the crosslinked collagen molecule.

U.S. Pat. No. 5,541,295 (Barrach) discloses monoclonal antibodies which bind specifically to Type II collagen, but not to its peptides, or vice versa. In particular, the following type II collagen sequences are included;

| | |
|---|---|
| GFQGL-Xaa-G-Xaa-Xaa-G-Xaa-Xaa-G | SEQ ID NO: 51 |
| GLQGL-Xaa-G-Xaa-Xaa-G-Xaa-SG | SEQ ID NO: 52 |

None of the above mentioned patents make reference to the cleavage of type II collagen by cathepsin K. Moreover, none of the patents disclose the usefulness of a quantitative measure of such fragments as biomarkers of subchondral bone remodelling.

References to collagen type II herein include specifically reference to human type II collagen.

The present invention now provides a method of assay to determine the extent of collagen type II resorption activity, or of degradation of mineralised cartilage, in a subject, comprising measuring in a biological sample from said subject fragments of collagen type II that contain a cathepsin K generated neo-epitope not shared by collagen type I by binding the neo-epitope with an immunological binding partner specific for the presence of said neo-epitope and detecting the level of binding of said binding partner.

Preferably, said extent of collagen type II resorption in said subject is evaluated by comparing the level of binding measured in said assay with levels previously established in healthy subjects and or in subjects having pathological collagen type II resorption activity, e.g. arthritis. Moreover the assay can provide an indication of the extent of subchondral bone remodelling activity and the comparator level used may be that seen in patients having a pathological level of subchondral bone remodelling.

The immunological binding partner is preferably specific for an epitope defined by one of the following amino acid sequences: . . . GQPGPA [symbol] SEQ ID NO:53; . . . EPGGVG [symbol] SEQ ID NO:54; [symbol] DQGVPG . . . SEQ ID NO:55; . . . PKGARG [symbol] SEQ ID NO:56; and [symbol] REGSPG . . . SEQ ID NO:57, wherein the symbol [symbol] indicates the end of the peptide chain generated by cathepsin K cleavage.

Preferably therefore, said immunological binding partner does not specifically bind a sequence as defined above if continued past the indicated cleavage site and does not bind intact collagen type II.

The assay may be conducted as a sandwich immunoassay using a second immunological binding partner which is specifically immunoreactive with a collagen type II amino acid sequence containing an isomerisation.

The invention includes an immunological binding partner against a C-terminal or N-terminal neo-epitope formed by proteinase cleavage of type II collagen which is not found in collagen type I.

Such an immunological binding partner preferably has specific binding affinity for a peptide having the N-terminal sequence [symbol] REGSPG . . . SEQ ID NO:57 or [symbol] DQGVPG . . . SEQ ID NO:55 or has specific binding affinity for a peptide having the C-terminal sequence . . . GQPGPA [symbol] SEQ ID NO:53; . . . EPGGVG [symbol] SEQ ID NO:54; or . . . PKGARG [symbol] SEQ ID NO:56.

The invention includes an immunoassay kit comprising an immunological binding partner as described together with at least one of calibration standards immunoreactive with said binding partner, a wash reagent, a buffer, a secondary immunological binding partner for revealing binding between said immunological binding partner of the invention and components of a sample, an enzyme label, an enzyme label substrate, a stopping reagent, or instructions for conducting an assay using said kit.

The biological sample may in particular be a body fluid sample and may be blood, serum, plasma, or urine.

As explained in WO02/095415, isomerisation of certain amino acids occurs naturally over time in proteins of the body, particularly at aspartic acid, asparagines, glutamic acid and glutamine residues, according to the illustrative reaction scheme:

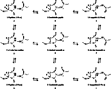

This may give rise to either or both of optical or structural isomerism at the affected residue and either can be recognised in a context specific manner (i.e. dependant on the presence of the appropriate flanking amino acid sequences) or context independent manner (i.e. not dependant on the presence of the appropriate flanking amino acid sequences) by a suitably selected immunological binding partner. In assays according to the invention a first immunological binding partner specifically binding a cathepsin K generated neo-epitope of collagen II may be used in combination with an immunological binding partner specifically binding an epitope containing a said isomerisation in a sandwich assay.

Assay formats useful in accordance with the present invention include both heterogeneous and homogeneous sandwich assay formats.

Homogeneous formats include the use of two different immunological binding partners bound to respective beads wherein the beads incorporate a detectable proximity signal activated when the beads are brought into proximity by their respective binding partners both binding to sites on a single fragment molecule.

Heterogeneous assay formats include those in which one of said immunological binding partners is immobilised to a solid support, said fragments are bound to said immobilised antibody and the binding of the other of said immunological binding partners to said fragments is detected.

Immunological binding partners for use in the present invention include whole antibodies, especially monoclonal antibodies, and antibody fragments with specific binding affinity. These include binding fragments such as Fab or F(ab′)₂.

The following procedure was used for identification of type II collagen sequences carrying Cathepsin K neoepitopes.

Human collagen type II (BIOCOL BC-3001) was dissolved in 10 mM acetic acid (400 μl added to 1 mg of collagen type II). Ten μg of procathepsin K (Calbiochem 342001) was activated by addition of 200 μl of 100 mM sodium acetate containing 10 mM DTT and 5 mM EDTA, pH 3.9 for 40 minutes at room temperature. Ten μg of MMP9 (Calbiochem 444231) was activated by addition of 200 μl of 1 mM APMA in DMSO for 2 hours at 37° C. For the Cathepsin K cleavage, 60 μl of collagen type II was added 120 μl of 50 mM sodium acetate, pH 5.5 containing 20 mM L-cysteine and 24 μl of activated cathepsin K for 4 hours at 37° C. For the MMP9 cleavage, 60 μl of collagen type II was added 120 μl of 100 mM Tris-HCl, 100 mM sodium chloride, 10 mM calcium chloride, 2 mM zinc chloride, pH 8.0 and 20 μl of MMP9 for 3 days at 37° C. The resulting proteolytic cleavage fragments were characterized by high performance liquid chromatography (HPLC)-tandem mass spectrometry (MS/MS) analysis. The MS/MS spectra were searched against protein databases using Sequest and X! Tandem database search algorithms. The following sequence hits of fragments were found for the Cathepsin K cleaved collagen type II:

| | |
|---|---|
| AQGPPGATGFPGAAGR | SEQ ID NO: 58 |
| ASGDRGPPGPV | SEQ ID NO: 59 |
| ASGDRGPPGPVGPPG | SEQ ID NO: 60 |
| GANGEKGEVGPPGPA | SEQ ID NO: 61 |
| GAPGEDGRPGPPGPQ | SEQ ID NO: 62 |
| GARGAPGERGETGPPGPA | SEQ ID NO: 63 |
| GDRGPPGPV | SEQ ID N0: 64 |
| GERGFPG | SEQ ID N0: 65 |
| GERGFPGER | SEQ ID NO: 66 |
| GESGSPGENGSPGPM | SEQ ID N0: 67 |
| GLPGPPGPPGEGGKPG | SEQ ID NO: 68 |
| GPIGPPGPA | SEQ ID NO: 69 |
| GPPGPPGKPGDDGEAGKPG | SEQ ID NO: 70 |
| GPPGPV | SEQ ID NO: 71 |
| GPPGPVGPA | SEQ ID NO: 72 |
| LPGPPGPPGEGGKPG | SEQ ID NO: 73 |
| NPGPPGPPGPPGPG | SEQ ID NO: 74 |
| PIGPP | SEQ ID NO: 75 |
| REGSPGADGPPGRDGAAGVK | SEQ ID NO: 76 |
| SNGNPGPPGPPGPS | SEQ ID NO: 77 |

Identified fragments were aligned with the sequence for human collagen type II (sp|P02458|CO2A1_HUAN Collagen alpha-1(II) chain), and cleavage sites were localized as indicated by the arrows.

SEQ ID NO: 78
QMAGGFDEKAGGAQLGVMQGPMGPMGPRGPPGPAGAPGPQGFQGNPGEPG
EPGVSGPMGPR [symbol] GPPGPPGKPGDDGEAGKPG [symbol] KAGERGPPGPQGARG
FPGTPGLPGVKGHRGYPGLDGAKGEAGAPGVK [symbol] GESGSPGENGSPGPM [symbol]
GPRGLPGERGRTGPAGAAGARGNDGQPGPA [symbol] GPPGPV [symbol] GPA [symbol] GGP
GFPGAPGAKGEAGPTGARGPEGAQGPRGEPGTPGSPGPAGASGNPGTD
GIPGAKGSAGAPGIAGAPGFPGPRGPPGPQGATGPLGPKGQTGEPGIAG
FKGEQGPKGEPGPAGPQGAPGPAGEEGKRGARGEPGGVG [symbol] PIGPP [symbol] G
ERGAPGNRGFPGQDGLAGPKGAPGERGPSGLAGPKGANGDPGRPGEPGL
PGARGLTGRPGDAGPQGKVGPS [symbol] GAPGEDGRPGPPGPQ [symbol] GARGQPGV
MGFPGPKGANGEPGKAGEKGLPGAPGLRGLPGKDGETGAAGPPGPAGPA
GERGEQGAPGPSGFQ [symbol] G [symbol] LPGPPGPPGEGGKPG [symbol] DQGVPGEAGAPG
LVGPR [symbol] GERGFPG [symbol] ER [symbol] GSPGAQGLQGPRGLPGTPGTDGPKGASGP
AGPPGAQGPPGLQGMPGERGAAGIAGPKGDRGDVGEKGPEGAPGKDGGRG
LT [symbol] GPIGPPGPA [symbol] GANGEKGEVGPPGPA [symbol] GSA [symbol] GARGAPGERGET
GPPGPA [symbol] GFAGPPGADGQPGAKGEQGEAGQKGDAGAPGPQGPSGAPGP
QGPTGVTGPKGARG [symbol] AQGPPGATGFPGAAGR [symbol] VGPPG [symbol] SNGNPGPP
GPPGPS [symbol] GKDGPKGARGDSGPPGRAGEPGLQGPAGPPGEKGEPGDDGP
SGAEGPPGPQGLAGQRGIVGLPGQRGERGFPGLPGPSGEPGKQGAPG [symbol]
AS [symbol] GDRGPPGPV [symbol] GPPG [symbol] LTGPAGEPG [symbol] REGSPGADGPPGRDGAA
GVK [symbol] GDRGETGAVGAPGAPGPPGSPGPAGPTGKQGDRGEAGAQGPMGP
SGPAGARGIQGPQGPRGDKGEAGEPGERGLKGHRGFTGLQGLPGPPGPS
GDQGASGPAGPSGPRGPPGPVGPSGKDGANGIPGPIGPPGPRGRSGETG
PAGPPG [symbol] NPGPPGPPGPPGPG [symbol] IDMSAFAGLGPREKGPDPLQYMRA

Preferred biomarker neoepitopes were selected based on protease and protein specificity. First protease specificity was assessed by comparison of Cathepsin K cleavage sites with cleavage sites of other proteases, e.g. MMP9 and MMP13, and cleavage sites that were in common were deselected. Next protein specificity of the remaining cleavage sites were assessed by identity search of 6 amino-terminal or 6 carboxy-terminal residues on either site of the cleavage site. The public available programme “Pattinprot” was used in the search of the UNIPROT/SWISSPROT databank. The preferred biomarker neoepitopes are indicated and listed below.

### Collagen α1 Chain Type II Collagen (1060 Residues)

SEQ ID NO: 78
QMAGGFDEKAGGAQLGVMQGPMGPMGPRGPPGPAGAPGPQGFQGNP
GEPGEPGVSGPMGPRGPPGPPGKPGDDGEAGKPGKAGERGPPGPQG
ARGFPGTPGLPGVKGHRGYPGLDGAKGEAGAPGVKGESGSPGENGS
PGPMGPRGLPGERGRTGPAGAAGARGNDGQPGPA [symbol] GPPGPVGPAGG
PGFPGAPGAKGEAGPTGARGPEGAQGPRGEPGTPGSPGPAGASGNP
GTDGIPGAKGSAGAPGIAGAPGFPGPRGPPGPQGATGPLGPKGQTG
EPGIAGFKGEQGPKGEPGPAGPQGAPGPAGEEGKRGARGEPGGVG [symbol]
PIGPPGERGAPGNRGFPGQDGLAGPKGAPGERGPSGLAGPKGANGD
PGRPGEPGLPGARGLTGRPGDAGPQGKVGPSGAPGEDGRPGPPGPQ
GARGQPGVMGFPGPKGANGEPGKAGEKGLPGAPGLRGLPGKDGETG
AAGPPGPAGPAGERGEQGAPGPSGFQGLPGPPGPPGEGGKPG [symbol] DQG
VPGEAGAPGLVGPRGERGFPGERGSPGAQGLQGPRGLPGTPGTDGP
KGASGPAGPPGAQGPPGLQGMPGERGAAGIAGPKGDRGDVGEKGPE
GAPGKDGGRGLTGPIGPPGPAGANGEKGEVGPPGPAGSAGARGAPG
ERGETGPPGPAGFAGPPGADGQPGAKGEQGEAGQKGDAGAPGPQGP
SGAPGPQGPTGVTGPKGARG [symbol] AQGPPGATGFPGAAGRVGPPGSNGN
PGPPGPPGPSGKDGPKGARGDSGPPGRAGEPGLQGPAGPPGEKGEP
GDDGPSGAEGPPGPQGLAGQRGIVGLPGQRGERGFPGLPGPSGEPG
KQGAPGASGDRGPPGPVGPPGLTGPAGEPG [symbol] REGSPGADGPPGRDG
AAGVKGDRGETGAVGAPGAPGPPGSPGPAGPTGKQGDRGEAGAQGP
MGPSGPAGARGIQGPQGPRGDKGEAGEPGERGLKGHRGFTGLQGLP
GPPGPSGDQGASGPAGPSGPRGPPGPVGPSGKDGANGIPGPIGPPG
PRGRSGETGPAGPPGNPGPPGPPGPPGPGIDMSAFAGLGPREKGPD
PLQYMRA

Preferred biomarker neoepitopes are marked in the above sequence and are extracted below.

### List of Preferred Biomarker Neoepitopes

| | | |
|---|---|---|
| 1 | . . . GQPGPA [symbol] | SEQ ID NO: 53 |
| 2 | . . . EPGGVG [symbol] | SEQ ID NO: 54 |
| 3 | [symbol] DQGVPG . . . | SEQ ID NO: 55 |
| 4 | . . . PKGARG [symbol] | SEQ ID NO: 56 |
| 5 | [symbol] REGSPG . . . | SEQ ID NO: 57 |

For the sequences of 1, 2, and 4 the immunological binding partner should preferably have specific binding affinity for peptide fragments comprising the C-terminal neoepitope.

For the sequences of 3 and 5 the immunological binding partner should preferably have specific binding affinity for peptide fragments comprising the N-terminal neoepitope.

The invention will be further described and illustrated by the following examples making reference to the accompanying drawings, in which:

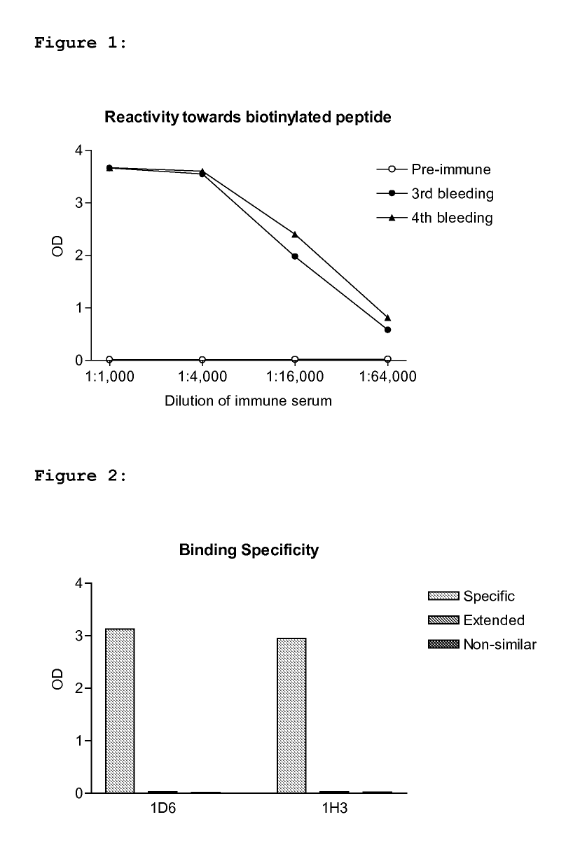

FIG. 1 shows antibody binding in sera studied in Example 1;

FIG. 2 shows monoclonal antibody binding observed in Example 1;

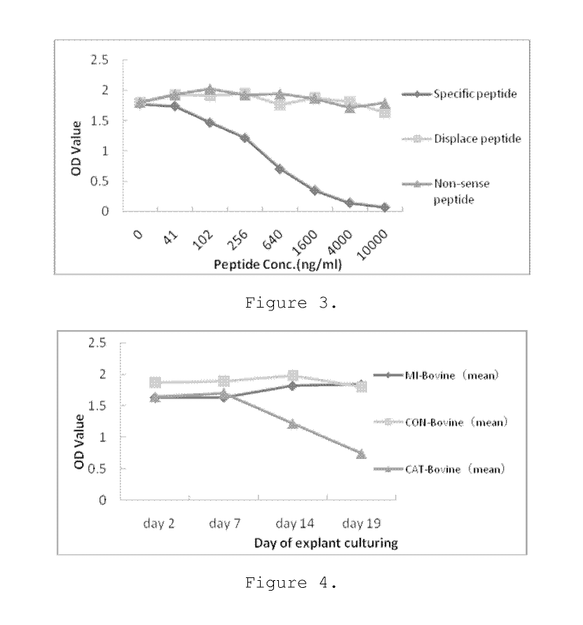

FIG. 3 shows monoclonal antibody binding observed in Example 3; and

FIG. 4 shows further monoclonal antibody binding observed in Example 3.

### EXAMPLE 1

### Generation of Monoclonal Antibodies Recognising a N-terminal Neoepitope

The following demonstrates that monoclonal antibodies can be generated against collagen II neo-epitopes. Synthetic peptides were prepared by standard techniques. To increase immunogenicity, the peptide (LTGPAGGGGC SEQ ID NO:78) was coupled at the C-terminus to the carrier protein KLH using site-directed coupling technology via the cysteine. Before immunisation, the immunogen was mixed 1:1 with Freund's Incomplete Adjuvant and the mixture was injected s.c. in Balb/c mice. The immunisation was repeated every 2 weeks for two months (four immunisations) and then continued with 4 weeks between each immunisation. Blood was obtained from the mice before immunisation initiated and one week after each immunization. The immune response was evaluated by testing the binding reactivity of mouse immune sera towards the C-terminal biotinylated synthetic peptide (LTGPAGEPGK-Biotin SEQ ID NO:79). The test for binding reactivity of mouse immune sera was based on binding of the immune serum to the biotinylated synthetic peptide that was bound to the surface of a streptavidin-coated microtitre plate. After incubation and washing the bound antibody was demonstrated by incubation with anti-mouse IgG conjugated to horseradish peroxidase, washing and addition of the chromogen TMB (FIG. 1).

Subsequent to attaining sufficient immune sera titers in the above mentioned screening test, the selected mice were rested for at least 4 weeks, and boosted i.p. withl immunogen without adjuvants. Three days later, the spleen was removed and used for fusion with myeloma cells using standard techniques. Antibodies from growing hybridomas were evaluated by their binding reactivity to the biotinylated synthetic peptide in the assay as described above. Additionally the cleavage specificity of the antibodies was demonstrated by minimum binding reactivity towards a one-residue extended coater (GLTGPAGEPGK-Biotin SEQ ID NO:80) as well as no binding towards a non-similar coater (Biotin-KGATGPLGPK SEQ ID NO:81) (FIG. 2).

### EXAMPLE 2

### Generation of Monoclonal Antibodies Recognising N- or C-terminal Neoepitopes

The following further demonstrates that monoclonal antibodies can be generated against cathepsin K mediated collagen type II neo-epitopes. To increase immunogenicity, the peptides EAGKPG [symbol] SEQ ID NO:82 (NB76), GQPGPA [symbol] SEQ ID NO:53 (NB77), EPGGVG [symbol] SEQ ID NO:54(NB78), [symbol] DQGVPG SEQ ID NO:55 (NB79), PKGARG [symbol] SEQ ID NO:56 (NB80) and [symbol] REGSPG SEQ ID NO:57 (NB81) were coupled at the N- or C-terminus as appropriate (i.e. were coupled at the end opposite the marked cleavage site) to the carrier protein KLH using site-directed coupling technology via a cysteine, which is added to the synthetic peptide.

Before immunisation, the immunogen was mixed 1:1 with Freund's Incomplete Adjuvant and the mixture was injected s.c. in Balb/c mice. The immunisation was repeated every 2 weeks for two months (four immunisations) and then continued with 4 weeks between each immunisation. Blood was obtained from the mice before immunisation was initiated and one week after each immunization. The immune response was evaluated by testing the binding reactivity of mouse immune sera towards the C-terminal biotinylated synthetic peptide corresponding to each of the selected sequences (e.g. REGSPGGADAP-Biotin SEQ ID NO:83). The test for binding reactivity of mouse immune sera was based on binding of the immune serum to the biotinylated synthetic peptide that was bound to the surface of a streptavidin-coated micro-titre plate. After incubation and washing the bound antibody was demonstrated by incubation with anti-mouse IgG conjugated to horseradish peroxidase, washing and addition of the chromogen TMB. Subsequent to attaining sufficient immune sera titers in the above mentioned screening test, the selected mice were rested for at least 4 weeks, and boosted i.p. with immunogen without adjuvants. Three days later, the spleen was removed and used for fusion with myeloma cells using standard techniques. Antibodies from growing hybridomas were evaluated by their binding reactivity to the biotinylated synthetic peptide in the assay as described above.

### EXAMPLE 3

### Testing Native Reactivity of a New Neoepitope Antibody

Several hybridomas and subsequent clones were selected for testing. The cleavage specificity of a typical one of these monoclonal antibodies (NB81) was demonstrated by showing minimal binding reactivity towards a one-residue extended peptide (GREGSPGGADAP SEQ ID NO:84) as well as no binding towards a non-similar peptide (KGATGPLGPK SEQ ID NO:85) in competition against a coater peptide REGSPGGADAP-Biotin SEQ ID NO:83 (FIG. 3). The signal was only displaced by the specific peptide (REGSPGGADAP SEQ ID NO:83) and it can therefore be concluded that the antibody is specific for the neoepitope.

Next the native reactivity of NB81 was tested using supernatants from bovine cartilage explants. Results are shown in FIG. 4. The release of fragments giving rise to assay signal could be inhibited by supernatants from cartilage when treated with catabolic cytokines for more than 7 days CAT-bovine). However this was neither observed with the non-catabolic control (CON-Bovine) nor a metabolically inactive control (MI-Bovine). (FIG. 4). This indicates that the fragment is related to cartilage turnover and degradation and it will therefore be a good marker for catK induced cartilage degradation.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

Kafienah W, Bromme D, Buttle D J, Croucher L J, Hollander A P. Human cathepsin K cleaves native type I and II collagens at the N-terminal end of the triple helix. Biochem J. 1998 May 1; 331 (Pt 3):727-32
Dejica V M, Mort J S, Layerty S, Percival M D, Antoniou J, Zukor D J, Poole A R. Cleavage of type II collagen by cathepsin K in human osteoarthritic cartilage. Am J. Pathol. 2008 July; 173(1):161-9. Epub 2008 May 29
Dean, D. D., Martel-Pelletier, J., Pelletier, J. P., Howell, D. S., and Woessner, J. F., Jr. 1989. Evidence for metalloproteinase and metalloproteinase inhibitor imbalance in human osteoarthritic cartilage. J Clin Invest 84:678-685.
Reboul, P., Pelletier, J. P., Tardif, G., Cloutier, J. M., and Martel-Pelletier, J. 1996. The new collagenase, collagenase-3, is expressed and synthesized by human chondrocytes but not by synoviocytes. A role in osteoarthritis. J Clin Invest 97:2011-2019.
Hui, W., Rowan, A. D., Richards, C. D., and Cawston, T. E. 2003. Oncostatin M in combination with tumor necrosis factor alpha induces cartilage damage and matrix metalloproteinase expression in vitro and in vivo. Arthritis Rheum 48:3404-3418.
Schaller, S., Henriksen, K., Hoegh-Andersen, P., Sondergaard, B. C., Sumer, E. U., Tanko, L. B., Qvist, P., and Karsdal, M. A. 2005. In vitro, ex vivo, and in vivo methodological approaches for studying therapeutic targets of osteoporosis and degenerative joint diseases: how biomarkers can assist? Assay. Drug Dev. Technol. 3:553-580.
Sumer, E. U., Schaller, S., Sondergaard, B. C., Tanko, L. B., and Qvist, P. 2006. Application of biomarkers in the clinical development of new drugs for chondroprotection in destructive joint diseases: a review. Biomarkers 11:485-506.
Christgau, S., Garnero, P., Fledelius, C., Moniz, C., Ensig, M., Gineyts, E., Rosenquist, C., and Qvist, P. 2001. Collagen type II C-telopeptide fragments as an index of cartilage degradation. Bone 29:209-215.
Mouritzen, U., Christgau, S., Lehmann, H. J., Tanko, L. B., and Christiansen, C. 2003. Cartilage turnover assessed with a newly developed assay measuring collagen type II degradation products: influence of age, sex, menopause, hormone replacement therapy, and body mass index. Ann. Rheum Dis 62:332-336.
Reijman, M., Hazes, J. M., Bierma-Zeinstra, S. M., Koes, B. W., Christgau, S., Christiansen, C., Uitterlinden, A. G., and Pols, H. A. 2004. A new marker for osteoarthritis: cross-sectional and longitudinal approach. Arthritis Rheum 50:2471-2478.
Hunter D J, Hart D, Snieder H, Bettica P, Swaminathan R, Spector T D. Evidence of altered bone turnover, vitamin D and calcium regulation with knee osteoarthritis in female twins. Rheumatology (Oxford). 2003A; 42:1311-16.
Hunter D J, Spector T D. The role of bone metabolism in osteoarthritis. Curr Rheumatol Rep. 2003B; 5:15-19.
Dieppe P, Cushnaghan J, Young P, Kirwan J. Prediction of the progression of joint space narrowing in osteoarthritis of the knee by bone scintigraphy. Ann Rheum Dis. 1993; 52:557-63.
Arden N, Nevitt M C. Osteoarthritis: epidemiology. Best Pract Res Clin Rheumatol. 2006; 20:3-25.
Burr D B, Radin E L. Microfractures and microcracks in subchondral bone: are they relevant to osteoarthrosis? Rheum Dis Clin North Am. 2003; 29:675-85.
Lajeunesse D, Reboul P. Subchondral bone in osteoarthritis: a biologic link with articular cartilage leading to abnormal remodeling. Curr Opin Rheumatol. 2003; 15:628-33.
Bailey A J, Mansell J P, Sims T J, Banse X. Biochemical and mechanical properties of subchondral bone in osteoarthritis. Biorheology. 2004; 41:349-58.
Woolf A D, Pfleger B: Burden of major musculoskeletal conditions. Bull World Health Organ 2003; 81:646-56
Lawrence R C, Helmick C G, Arnett F C et al. Estimates of the prevalence of arthritis nad selected musculoskeletal disorders in the United States. Arthritis Rheum 1998; 41:778-99.
Pritzker K P, Gay S, Jimenez S A, Ostergaard K, Pelletier J P, Revell P A, Salter D, van den Berg W B. Osteoarthritis cartilage histopathology: grading and staging. Osteoarthritis Cartilage 2006; 14:13-29.
Birmingham J D, Vilim V, Kraus V B. Collagen biomarkers for arthritis applications. Biomarker Insights 2006; 2: 61-76.
Fraser A, Fearon U, Billinghurst R C, Ionescu M, Reece R, Barwick T, Emery P, Poole A R, Veale D J. Turnover of type II collagen and aggrecan in cartilage matrix at the onset of inflammatory arthritis in humans: relationship to mediators of systemic and local inflammation. Arthritis Rheum. 2003 November; 48(11):3085-95.
Billinghurst R C, Dahlberg L, Ionescu M, Reiner A, Bourne R, Rorabeck C, Mitchell P, Hambor J, Diekmann O, Tschesche H, Chen J, Van Wart H, Poole A R. Enhanced cleavage of type II collagen by collagenases in osteoarthritic articular cartilage. J Clin Invest. 1997 Apr. 1; 99(7):1534-45.
Poole A R, Ionescu M, Fitzcharles M A, Billinghurst R C. The assessment of cartilage degradation in vivo: development of an immunoassay for the measurement in body fluids of type II collagen cleaved by collagenases. J Immunol Methods. 2004 November; 294(1-2):145-53.
Otterness I G, Downs J T, Lane C, Bliven M L, Stukenbrok H, Scampoli D N, Milici A J, Mezes P S. Detection of collagenase-induced damage of collagen by 9A4, a monoclonal C-terminal neoepitope antibody. Matrix Biol. 1999 August; 18(4):331-41.
Downs J T, Lane C L, Nestor N B, McLellan T J, Kelly M A, Karam G A, Mezes P S, Pelletier J P, Otterness I G. Analysis of collagenase-cleavage of type II collagen using a neoepitope ELISA. J Immunol Methods. 2001 Jan. 1; 247(1-2):25-34.

1. A method of assay to determine the extent of collagen type II resorption activity in a subject, comprising measuring in a biological sample from said subject fragments of collagen type II that contain a cathepsin K generated neo-epitope not shared by collagen type I by binding the neo-epitope with an immunological binding partner specific for the presence of said neo-epitope and detecting the level of binding of said binding partner.

2. A method as claimed in claim 1, wherein said extent of collagen type II resorption in said subject is evaluated by comparing the level of binding measured in said assay with levels previously established in healthy subjects and or in subjects having pathological collagen type II remodelling activity.

3. A method as claimed in claim 2, wherein said pathological collagen type II resorption activity is arthritis.

4. A method as claimed in claim 1 for determining the extent of subchondral bone remodelling activity.

5. A method as claimed in claim 1, wherein said extent of subchondral bone remodelling in said subject is evaluated by comparing the level of binding measured in said assay with levels previously established in healthy subjects and or in subjects having pathological subchondral bone remodelling activity.

6. A method as claimed in claim 1, wherein the immunological binding partner is specific for an epitope defined by one of the following amino acid sequences: . . . GQPGPA [symbol] SEQ ID NO:53; . . . EPGGVG [symbol] SEQ ID NO:54; [symbol] DQGVPG . . . SEQ ID NO:55; . . . . PKGARG [symbol] SEQ ID NO:56; and [symbol]
REGSPG . . . SEQ ID NO:57, wherein the symbol [symbol] indicates the end of the peptide chain generated by cathepsin K cleavage.

7. A method as claimed in claim 6, wherein said immunological binding partner does not specifically bind a sequence as defined in claim 4 if continued past the indicated cleavage site.

8. A method as claimed in claim 1, conducted as a sandwich immunoassay using a second immunological binding partner which is specifically immunoreactive with a collagen type II amino acid sequence containing an isomerisation.

9. An immunological binding partner against a C-terminal or N-terminal neo-epitope formed by proteinase cleavage of type II collagen.

10. An immunological binding partner having specific binding affinity for a peptide having the N-terminal sequence [symbol] REGSPG . . . SEQ ID NO:57 or [symbol] DQGVPG . . . SEQ ID NO:55.

11. An immunological binding partner having specific binding affinity for a peptide having the C-terminal sequence . . . GQPGPA [symbol] SEO ID NO:53; . . . EPGGVG [symbol] SEQ ID NO:54; or . . . PKGARG [symbol] SEQ ID NO:56.

12. An immunoassay kit comprising an immunological binding partner as claimed in claim 9 together with at least one of calibration standards immunoreactive with said binding partner, a wash reagent, a buffer, a secondary immunological binding partner for revealing binding between said immunological binding partner as claimed in claim 6 and components of a sample, an enzyme label, an enzyme label substrate, a stopping reagent, or instructions for conducting an assay using said kit.


SEQUENCE LISTING (85 sequences)

SEQ ID NO: 1 - protein, 8 aa, Homo sapiens
  MISC_FEATURE (4)..(4): Hydroxyproline
EGPPGPQG

SEQ ID NO: 2 - protein, 7 aa, Homo sapiens
  MISC_FEATURE (3)..(3): Hydroxyproline
GPPGPQG

SEQ ID NO: 3 - protein, 7 aa, Homo sapiens
GPPGPQG

SEQ ID NO: 4 - protein, 20 aa, Bos taurus
PGDDGEAGKPGKSGERGPPG

SEQ ID NO: 5 - protein, 20 aa, Homo sapiens
PGDDGEAGKPGKAGERGPPG

SEQ ID NO: 6 - protein, 7 aa, Homo sapiens
GEAGKPG

SEQ ID NO: 7 - protein, 7 aa, Homo sapiens
GPPGPQG

SEQ ID NO: 8 - protein, 10 aa, Homo sapiens
GEPGDDGPSG

SEQ ID NO: 9 - protein, 13 aa, Homo sapiens
APGEDGRPGPPGP

SEQ ID NO: 10 - protein, 16 aa, Homo sapiens
GKVGPSGAPGEDGRPG

SEQ ID NO: 11 - protein, 9 aa, Homo sapiens
AEGPPGPQG

SEQ ID NO: 12 - protein, 10 aa, Homo sapiens
GPPGPQGLAG

SEQ ID NO: 13 - protein, 9 aa, Homo sapiens
GEPGDDGPS

SEQ ID NO: 14 - protein, 16 aa, Homo sapiens
GEPGDDGPSGAEGPPG

SEQ ID NO: 15 - protein, 21 aa, Homo sapiens
EKGEPGDDAPSGAEGPPGPQG

SEQ ID NO: 16 - protein, 21 aa, Homo sapiens
GPPGPPGKPGDDGEAGKPGKA

SEQ ID NO: 17 - protein, 21 aa, Homo sapiens
GPPGPRGRSGETGPAGPPGNP

SEQ ID NO: 18 - protein, 22 aa, Homo sapiens
GAPGPQGFQGNPGEPGEPGVSY

SEQ ID NO: 19 - protein, 20 aa, Homo sapiens
GEPGDDAGPSGAEGPPGPQG

SEQ ID NO: 20 - protein, 9 aa, Homo sapiens
HRGYPGLDG

SEQ ID NO: 21 - protein, 23 aa, Homo sapiens
CGKVGPSGAPGEDGRPGPPGPQY

SEQ ID NO: 22 - protein, 13 aa, Homo sapiens
APGEDGRPGPPGP

SEQ ID NO: 23 - protein, 4 aa, Homo sapiens
GQPG

SEQ ID NO: 24 - protein, 11 aa, Homo sapiens
CGGEGPPGPQG

SEQ ID NO: 25 - protein, 19 aa, Homo sapiens
GAEGPPGPQGLAGQRGIVG

SEQ ID NO: 26 - protein, 19 aa, Homo sapiens
GAPGTPGPQGIAGQRGVVG

SEQ ID NO: 27 - protein, 19 aa, Homo sapiens
GPPGTPGPQGLLGAPGILG

SEQ ID NO: 28 - protein, 19 aa, Homo sapiens
GPPGAPGPLGIAGITGARG

SEQ ID NO: 29 - protein, 12 aa, Homo sapiens
CGGEGPPGPQGL

SEQ ID NO: 30 - protein, 13 aa, Homo sapiens
CGGEGPPGPQGLA

SEQ ID NO: 31 - protein, 10 aa, Homo sapiens
CGGEGPPGPQ

SEQ ID NO: 32 - protein, 9 aa, Homo sapiens
CGGEGPPGP

SEQ ID NO: 33 - protein, 8 aa, Homo sapiens
CGPPGPQG

SEQ ID NO: 34 - protein, 30 aa, Homo sapiens
LQGPAGPPGEKGEPGDDGPSGAEGPPGPQG

SEQ ID NO: 35 - protein, 5 aa, Homo sapiens
PGPQG

SEQ ID NO: 36 - protein, 6 aa, Homo sapiens
PPGPQG

SEQ ID NO: 37 - protein, 31 aa, Homo sapiens
VLQGPAGPPGEKGEPGDDGPSGAEGPPGPQG

SEQ ID NO: 38 - protein, 47 aa, Homo sapiens
KGARGDSGPPGRAGEPGLQGPAGPPGEKGEPGDDGPSGAEGPPGPQG

SEQ ID NO: 39 - protein, 45 aa, Homo sapiens
ARGDSGPPGRAGEPGLQGPAGPPGEKGEPGDDGPSGAEGPPGPQG

SEQ ID NO: 40 - protein, 28 aa, Homo sapiens
GPAGPPGEKGEPGDDGPSGAEGPPGPQG

SEQ ID NO: 41 - protein, 145 aa, Homo sapiens
GPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLGLPGKDGETGAEGPPPA

SEQ ID NO: 42 - protein, 347 aa, Homo sapiens
MPGERGAAGIAGPKGDRGDVGEKGPEGAPGKDGGRGLTGPIGPPGPAGANGEKGEVGPPGPAGSAGARGAPGERGETGPPGTSGIAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGQQGAPGASGDRGPPGPVGPPGLTGPAGEPGREGSPGADGPPGRDGAAGVKGDRGETGAVGAPGAPGPPGSPGPAGPTGKQGDRGEAGAQGPM

SEQ ID NO: 43 - protein, 6 aa, Homo sapiens
EKGPDP

SEQ ID NO: 44 - protein, 7 aa, Homo sapiens
  MISC_FEATURE (4)..(5): Isomerised peptide bond
GDIKDIV

SEQ ID NO: 45 - protein, 5 aa, Homo sapiens
  MISC_FEATURE (4)..(5): Isomerised peptide bond
EKGPD

SEQ ID NO: 46 - protein, 7 aa, Homo sapiens
GDIKDIV

SEQ ID NO: 47 - protein, 6 aa, Homo sapiens
EKGPDP

SEQ ID NO: 48 - protein, 5 aa, Homo sapiens
PGPKG

SEQ ID NO: 49 - protein, 6 aa, Homo sapiens
PGVKGE

SEQ ID NO: 50 - protein, 6 aa, Homo sapiens
GQKGEP

SEQ ID NO: 51 - protein, 13 aa, Homo sapiens
  MISC_FEATURE (6)..(6): Unknown amino acid
GFQGLXGXXGXXG

SEQ ID NO: 52 - protein, 13 aa, Homo sapiens
  MISC_FEATURE (6)..(6): Unspecified amino acid
GLQGLXGXXGXSG

SEQ ID NO: 53 - protein, 6 aa, Homo sapiens
GQPGPA

SEQ ID NO: 54 - protein, 6 aa, Homo sapiens
EPGGVG

SEQ ID NO: 55 - protein, 6 aa, Homo sapiens
DQGVPG

SEQ ID NO: 56 - protein, 6 aa, Homo sapiens
PKGARG

SEQ ID NO: 57 - protein, 6 aa, Homo sapiens
REGSPG

SEQ ID NO: 58 - protein, 16 aa, Homo sapiens
AQGPPGATGFPGAAGR

SEQ ID NO: 59 - protein, 11 aa, Homo sapiens
ASGDRGPPGPV

SEQ ID NO: 60 - protein, 15 aa, Homo sapiens
ASGDRGPPGPVGPPG

SEQ ID NO: 61 - protein, 15 aa, Homo sapiens
GANGEKGEVGPPGPA

SEQ ID NO: 62 - protein, 15 aa, Homo sapiens
GAPGEDGRPGPPGPQ

SEQ ID NO: 63 - protein, 18 aa, Homo sapiens
GARGAPGERGETGPPGPA

SEQ ID NO: 64 - protein, 9 aa, Homo sapiens
GDRGPPGPV

SEQ ID NO: 65 - protein, 7 aa, Homo sapiens
GERGFPG

SEQ ID NO: 66 - protein, 9 aa, Homo sapiens
GERGFPGER

SEQ ID NO: 67 - protein, 15 aa, Homo sapiens
GESGSPGENGSPGPM

SEQ ID NO: 68 - protein, 16 aa, Homo sapiens
GLPGPPGPPGEGGKPG

SEQ ID NO: 69 - protein, 9 aa, Homo sapiens
GPIGPPGPA

SEQ ID NO: 70 - protein, 19 aa, Homo sapiens
GPPGPPGKPGDDGEAGKPG

SEQ ID NO: 71 - protein, 6 aa, Homo sapiens
GPPGPV

SEQ ID NO: 72 - protein, 9 aa, Homo sapiens
GPPGPVGPA

SEQ ID NO: 73 - protein, 15 aa, Homo sapiens
LPGPPGPPGEGGKPG

SEQ ID NO: 74 - protein, 14 aa, Homo sapiens
NPGPPGPPGPPGPG

SEQ ID NO: 75 - protein, 5 aa, Homo sapiens
PIGPP

SEQ ID NO: 76 - protein, 20 aa, Homo sapiens
REGSPGADGPPGRDGAAGVK

SEQ ID NO: 77 - protein, 14 aa, Homo sapiens
SNGNPGPPGPPGPS

SEQ ID NO: 78 - protein, 10 aa, Homo sapiens
LTGPAGGGGC

SEQ ID NO: 79 - protein, 10 aa, Homo sapiens
LTGPAGEPGK

SEQ ID NO: 80 - protein, 11 aa, Homo sapiens
GLTGPAGEPGK

SEQ ID NO: 81 - protein, 10 aa, Homo sapiens
KGATGPLGPK

SEQ ID NO: 82 - protein, 6 aa, Homo sapiens
EAGKPG

SEQ ID NO: 83 - protein, 11 aa, Homo sapiens
REGSPGGADAP

SEQ ID NO: 84 - protein, 12 aa, Homo sapiens
GREGSPGGADAP

SEQ ID NO: 85 - protein, 10 aa, Homo sapiens
KGATGPLGPK